(19) United States
(12) Patent Application Publication
(10) Pub. No.: US 2026/0041862 A1
(43) Pub. Date: Feb. 12, 2026

(54) Positive expiratory pressure device with flow and oscillation control

(71) Applicant: Mercury Enterprises, Inc., Clearwater, FL (US)

(72) Inventor: Allen Raymond, Clearwater, FL (US)

(21) Appl. No.: 18/800,405

(22) Filed: Aug. 12, 2024

(51) Int. Cl. A61M 16/00; A61M 16/10; A61M 16/20

(52) U.S. Cl. CPC A61M 16/0003; A61M 16/10; A61M 16/201; A61M 2016/0027; A61M 2202/0007; A61M 2202/0208; A61M 2205/3327; A61M 2205/3331

(57) ABSTRACT

The positive expiratory pressure device with flow and oscillation control offers improved control over both airflow and oscillation characteristics. The primary mechanical elements of the device include an oscillation mechanism that creates intermittent airflow occlusion during exhalation. This mechanism is coupled with an adjustment system that allows for precise control over the oscillation frequency. Another significant feature of the invention is an integrated air manifold system. The air manifold provides adjustable control over the airflow through the device. By adjusting the valve within the manifold, a user can modify the overall resistance to exhalation, thereby controlling the amount of back-pressure generated during therapy. This feature allows for a wider range of pressure settings than a static manifold, making a device suitable for use for patients with varying lung capacities and conditions.

### FIELD

This invention relates to the field of respiratory treatment device and more particularly to a positive expiratory pressure device.

### BACKGROUND

Positive Expiratory Pressure (PEP) therapy is a respiratory treatment used to improve lung function and facilitate airway clearance in patients with various respiratory conditions. PEP devices are medical apparatuses designed to provide resistance during exhalation, creating back pressure in the airways that helps keep the airways open and promotes the mobilization of secretions.

PEP therapy has been utilized for several decades, with early devices consisting of simple mechanical valves that provided a fixed resistance to exhalation. These devices have proven beneficial in treating conditions such as chronic obstructive pulmonary disease (COPD), cystic fibrosis, bronchiectasis, and post-operative pulmonary complications.

As the field has advanced, more sophisticated PEP devices have been developed, including oscillating PEP (OPEP) devices. OPEP devices combine the benefits of traditional PEP therapy with oscillations or vibrations in the airflow, which enhances mucus clearance and lung recruitment.

While existing PEP and OPEP devices have shown clinical efficacy, there remains a need for improved designs that offer greater control over therapy parameters, enhanced ease of use, and better adaptability to individual patient needs.

### SUMMARY

The positive expiratory pressure device with flow and oscillation control offers improved control over both airflow and oscillation characteristics. This novel device includes several key features that work in concert to provide a more effective and customizable respiratory therapy experience.

The primary mechanical elements of the device include an oscillation mechanism that creates intermittent airflow occlusion during exhalation. This mechanism is coupled with an adjustment system that allows for control over the oscillation frequency. Specifically, the device incorporates a slider mechanism that can be manipulated by the user or physician to limit the range of motion of the oscillation mechanism. By adjusting the slider, which is operatively connected to the oscillation mechanism, the user can increase or decrease the frequency of oscillations, tailoring the therapy to their specific needs.

Another significant feature of the invention is an integrated air manifold system. The air manifold provides adjustable control over the airflow through the device. By adjusting the valve within the manifold, a user can modify the overall resistance to exhalation, thereby controlling the amount of back-pressure generated during therapy. This feature allows for a wider range of pressure settings than a static manifold, making a device suitable for use for patients with varying lung capacities and conditions.

The combination of the adjustable oscillation mechanism and the controllable air manifold results in a highly versatile PEP device. Users can independently adjust both the frequency of oscillations and the level of expiratory resistance, allowing for a personalized therapy regimen. This level of customization of the single device addresses the diverse needs of patients with different respiratory conditions, different breathing issues, or at various stages of treatment.

An oxygen port allows for the connection of an oxygen source.

The device is designed with user-friendliness in mind. The adjustment mechanisms are readily accessible and operable without disassembly.

As an additional feature, the device optionally includes a manometer. The manometer is located at the distal end of the housing—the opposite end of the housing with respect to the mouthpiece—placing it in a position that is readily visible to the physician or clinician. This simplifies the process of reviewing a patient's progress and performance.

In summary, this invention represents a significant advancement in PEP device technology, offering unprecedented levels of control over both airflow and oscillation parameters. Its innovative design addresses the limitations of existing devices and provides a more adaptable, effective solution for respiratory therapy.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric view of the positive expiratory pressure device with flow and oscillation control.

FIG. 2 illustrates an exploded view of the positive expiratory pressure device with flow and oscillation control.

FIG. 3 illustrates an exploded view of the valve manifold and rocker mechanism of the positive expiratory pressure device with flow and oscillation control.

FIG. 4 illustrates a first cross-sectional view of the positive expiratory pressure device with flow and oscillation control.

FIG. 5 illustrates a second cross-sectional view of the positive expiratory pressure device with flow and oscillation control.

FIG. 6 illustrates a third cross-sectional view of the positive expiratory pressure device with flow and oscillation control.

FIG. 7 illustrates a cross-sectional view showing the inhalation flow path of the positive expiratory pressure device with flow and oscillation control.

FIG. 8 illustrates a cross-sectional view showing the exhalation flow path of the positive expiratory pressure device with flow and oscillation control.

FIG. 9 illustrates a view of the oscillating mechanism in a closed position of the positive expiratory pressure device with flow and oscillation control.

FIG. 10 illustrates a view of the oscillating mechanism in an open position of the positive expiratory pressure device with flow and oscillation control.

FIG. 11 illustrates a first cross-sectional view of the air manifold and oscillating mechanism of the positive expiratory pressure device with flow and oscillation control.

FIG. 12 illustrates a second cross-sectional view of the air manifold and oscillating mechanism of the positive expiratory pressure device with flow and oscillation control.

FIG. 13 illustrates a first view of the oscillating mechanism and frequency adjustment slider of the positive expiratory pressure device with flow and oscillation control.

FIG. 14 illustrates a second view of the oscillating mechanism and frequency adjustment slider of the positive expiratory pressure device with flow and oscillation control.

FIG. 15 illustrates a third view of the oscillating mechanism and frequency adjustment slider of the positive expiratory pressure device with flow and oscillation control.

FIG. 16 illustrates a second isometric view showing the optional nebulizer of the positive expiratory pressure device with flow and oscillation control.

FIG. 17 illustrates a fourth cross-sectional view showing the optional nebulizer of the positive expiratory pressure device with flow and oscillation control.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

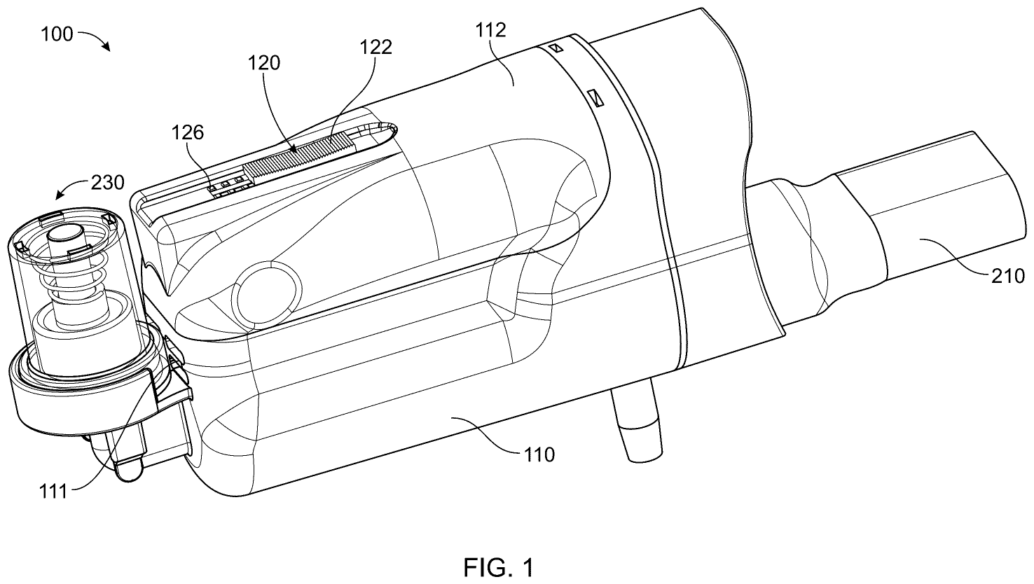

Referring to FIG. 1, a first isometric view of the positive expiratory pressure device with flow and oscillation control is shown.

The positive expiratory pressure device with flow and oscillation control 100 is shown with bottom cover 110 and top cover 112.

The oscillation frequency adjustment mechanism 120 is partially visible, with the slider 122 shown and protrusion 126 that interface with detents on the underside of the slider 122, holding the slider 122 in the selected position.

Ambient air ports 111 are visible in the bottom cover 110.

Manometer 230, measuring the patient's exhalation pressure, is placed at the far end of the positive expiratory pressure device with flow and oscillation control 100 where it is readily visible by a physician or clinician.

Also shown is mouth piece 210.

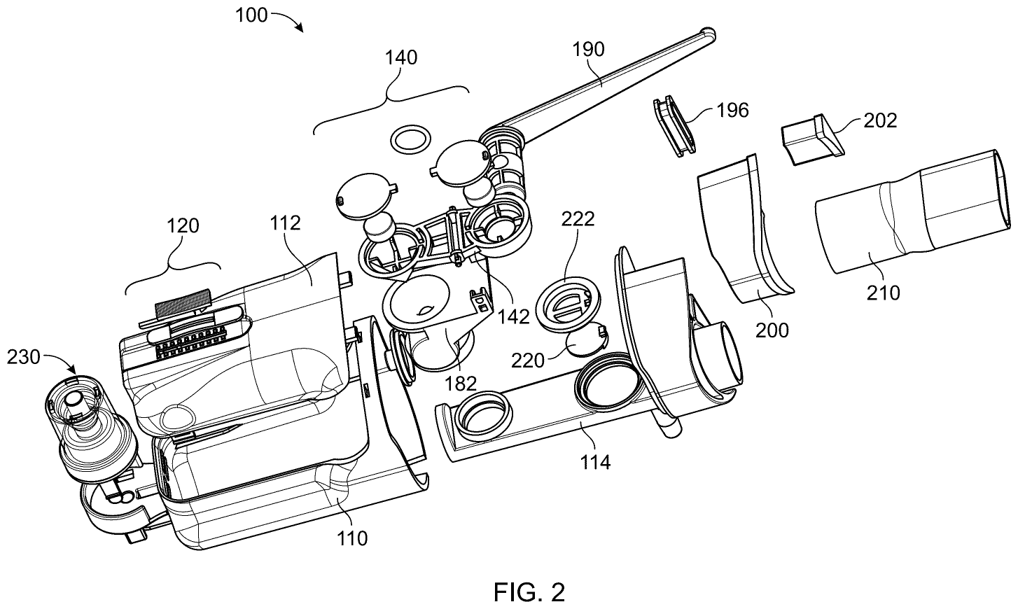

Referring to FIG. 2, an exploded view of the positive expiratory pressure device with flow and oscillation control is shown.

The positive expiratory pressure device with flow and oscillation control 100 is shown in an exploded view. The bottom cover 110 is shown separated from the top cover 112, with the oscillation frequency adjustment mechanism 120 primarily associated with the top cover 112.

The oscillation mechanism 140 with rocker arm 142 sits on top of the air manifold 182.

The flow control lever 190 passes through the flow control lever seal 196 to limit air leakage.

The body 114 includes the one-way valve 220 with one-way valve retaining mechanism 222, the one-way valve 220 allowing air to pass during inhalation but blocking the flow of air during exhalation.

Filler cap large 200 and filler cap small 202 fill extra holes in the housing to prevent leakage of air.

The mouthpiece 210 is placed within the user's mouth, through which the user will both inhale and exhale. The manometer 230 measures the exhalation pressure created by the user.

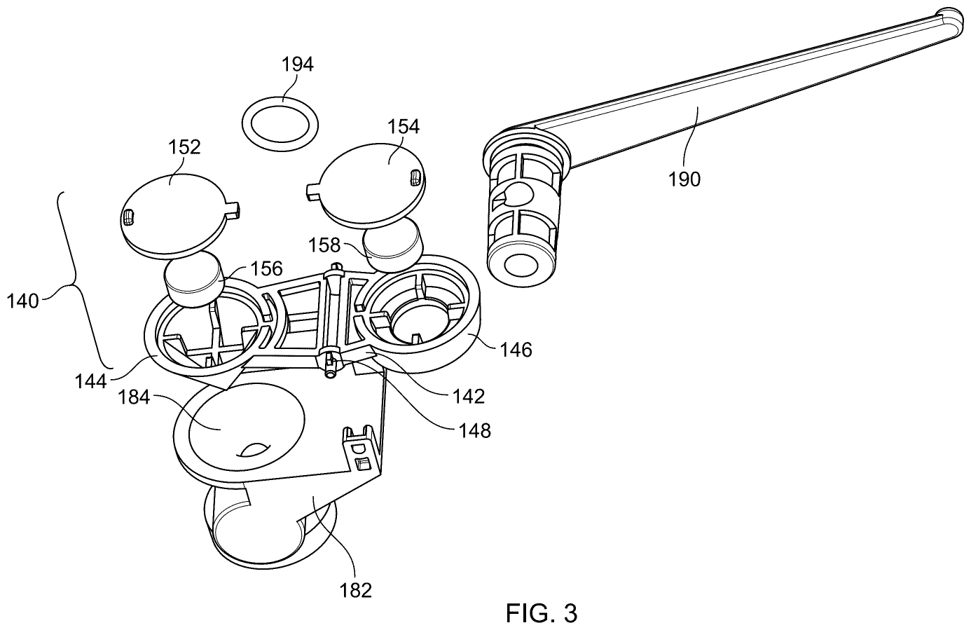

Referring to FIG. 3, an exploded view of the valve manifold and rocker mechanism of the positive expiratory pressure device with flow and oscillation control is shown.

The oscillation mechanism 140 rapidly opens and closes during exhalation to alternate between restricting and permitting the passage of air.

This is accomplished by the pivoting movement of the rocker arm 142, the plugging end 144 moving toward and away from the conical exhalation port 184 of the air manifold 182, repeatedly stopping and starting the flow of air.

The rocker arm 142 includes a plugging end 144 and a counterweight end 146, rotating with respect to a fulcrum 148.

A first rocker arm cap 152 covers a first oscillator weight 156 within the plugging end 144, and a second rocker arm cap 154 covers a second oscillator weight 158 within the counterweight end 146.

The flow control lever 190 fits into the air manifold 182, with air leakage prevented by the O-ring 194. The flow control lever 190 is accessible from outside the housing, allowing for adjustment by the user or physician.

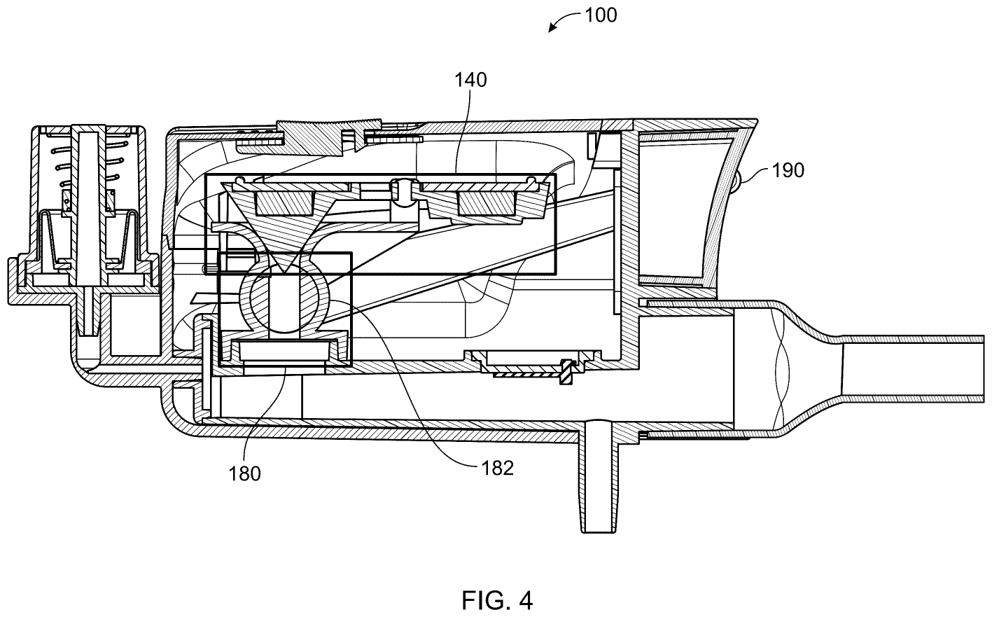

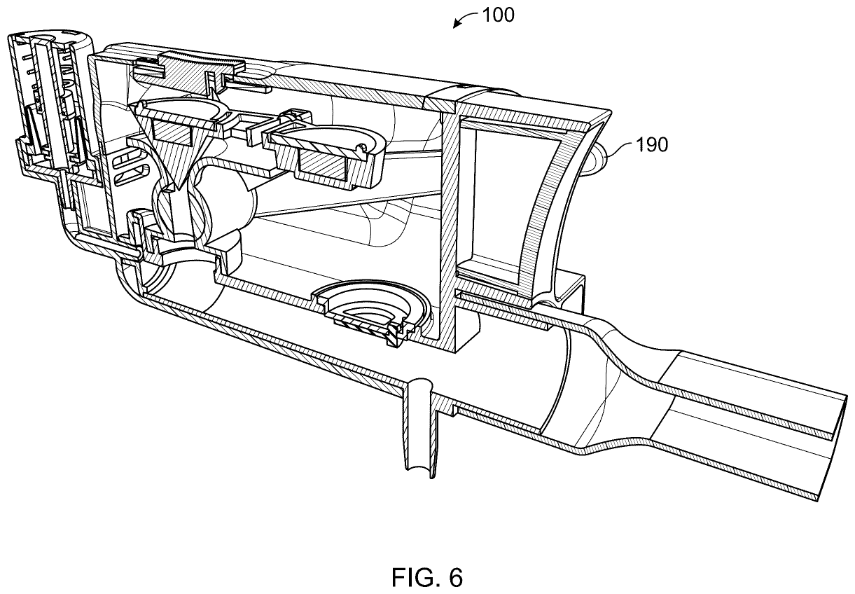

Referring to FIG. 4-6, first, second, and third cross-sectional views of the positive expiratory pressure device with flow and oscillation control are shown.

The oscillation mechanism 140 and the flow control mechanism 180 are related and share components.

The flow control lever 190 is shown passing through the flow control lever seal 196 of the positive expiratory pressure device with flow and oscillation control 100. The flow control lever 190 begins at the air manifold 182 and ends at the exterior of the device where it is accessible by the user.

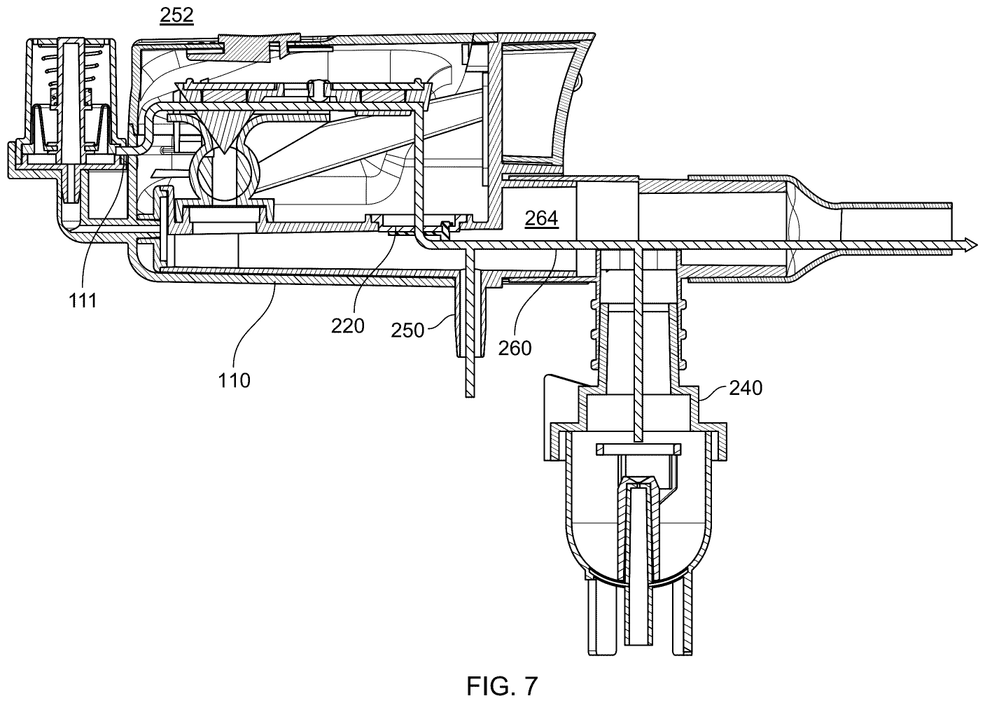

Referring to FIG. 7, a cross-sectional view showing the inhalation flow path of the positive expiratory pressure device with flow and oscillation control is shown.

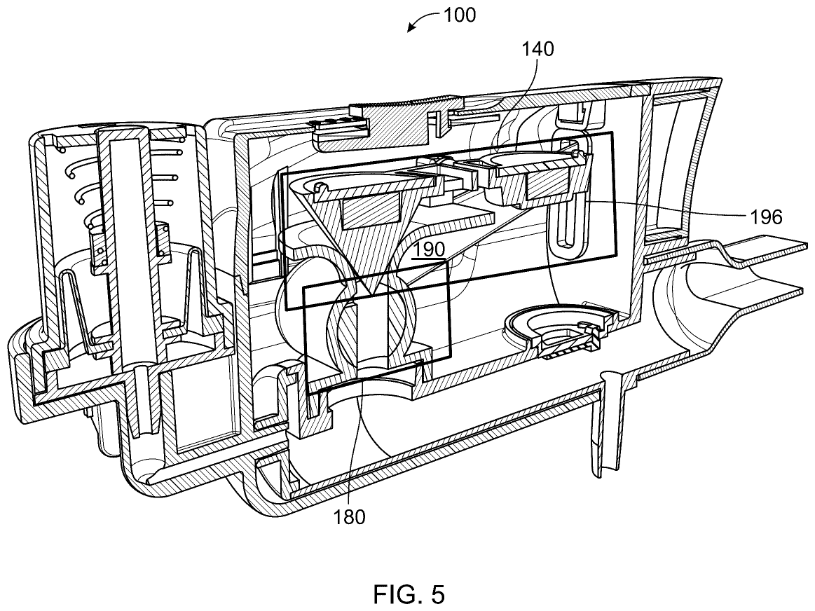

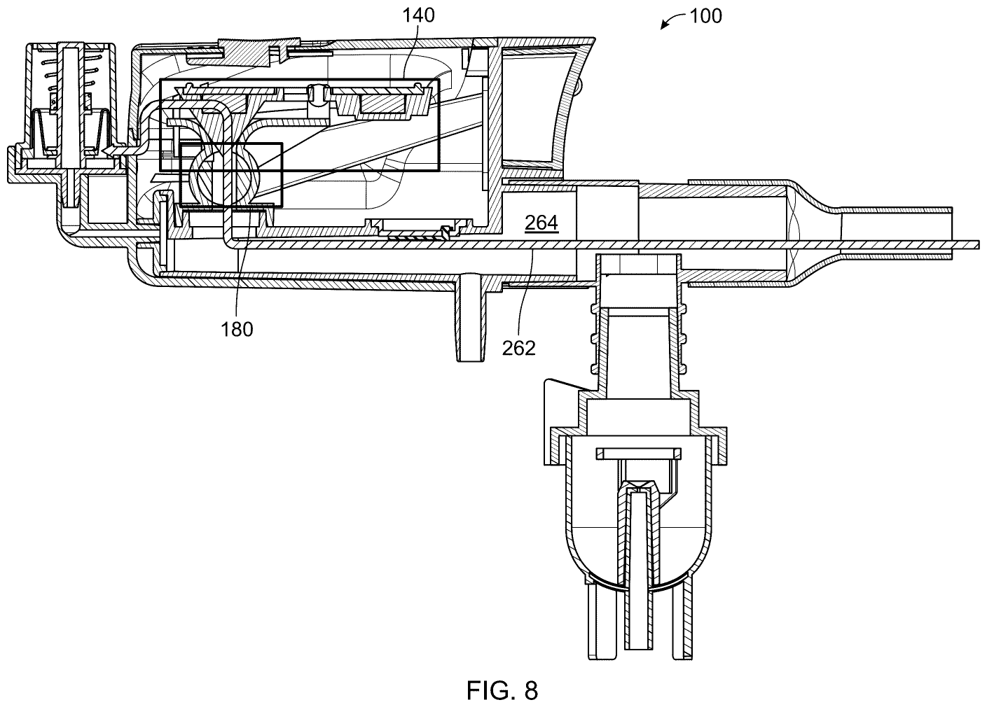

The inhalation flow path 260 has three inputs: ambient air 252; the oxygen port 250; and the optional nebulizer 240. Inhalation flow path 260 flows into the bottom cover 110 via the ambient air port 111, through the one-way valve 220 and the airflow pathway 264, optionally receiving input from the oxygen port 250 and/or the nebulizer 240, and bypassing the oscillation mechanism 140 and flow control mechanism 180 (see FIG. 5). Referring to FIG. 8, a cross-sectional view showing the exhalation flow path of the positive expiratory pressure device with flow and oscillation control is shown.

The exhalation flow path 262 follows the airflow pathway 264, then flowing through the flow control mechanism 180 and the oscillation mechanism 140 before exiting the positive expiratory pressure device with flow and oscillation control 100.

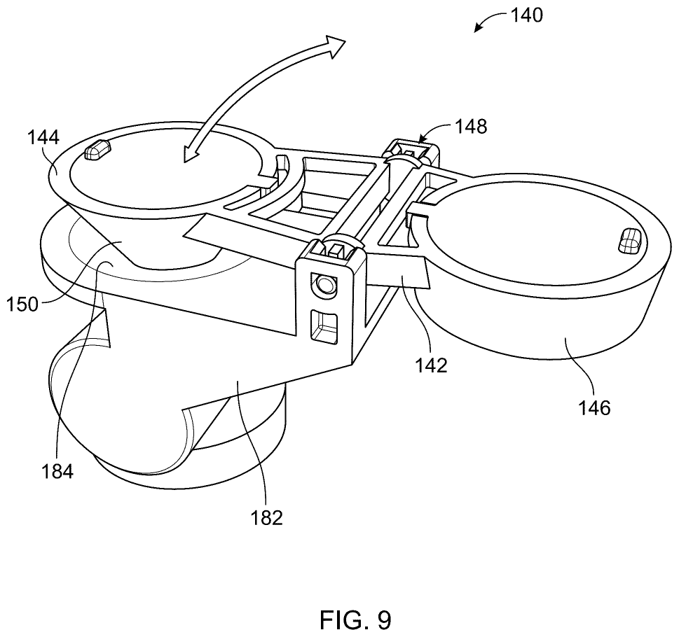

Referring to FIG. 9, a view of the oscillating mechanism in a closed position of the positive expiratory pressure device with flow and oscillation control is shown.

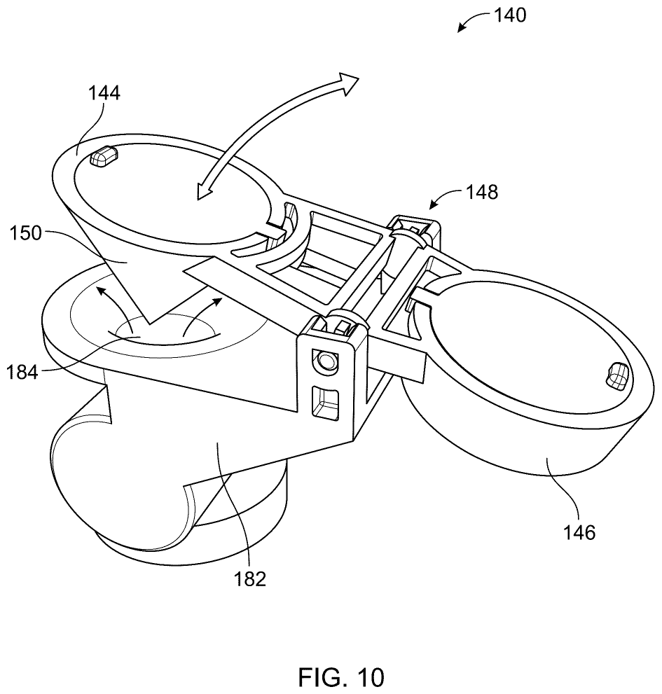

In the closed position, the plugging end 144 of the oscillation mechanism 140 is seated within the conical exhalation port 184 of the air manifold 182, preventing the flow of exhaled air. The pressure of the air against the plugging end 144 causes upward motion, aided by the counterweight end 146 of the rocker arm 142 rotating across the fulcrum 148. This causes conical plug 150 to rise out of the conical exhalation port 184, transitioning to the open position shown in FIG. 10.

Referring to FIG. 10, a view of the oscillating mechanism in an open position of the positive expiratory pressure device with flow and oscillation control is shown.

Shown in the open position, the plugging end 144 of the oscillation mechanism 140 is raised, the conical plug 150 allowing air to pass out of the conical exhalation port 184 of the air manifold 182. The counterweight end 146 has rotated downward, again rotating across the fulcrum 148. The longer lever arm of the plugging end 144 as compared to the counterweight end 146, in combination with the selection of first oscillator weight 156 compared to the second oscillator weight 158 (see FIG. 3) causes the conical plug 150 to descend from this upper position toward the conical exhalation port 184. The oscillation mechanism 140 then reaches the closed position shown in FIG. 9, the process repeating itself in a cyclical oscillating fashion.

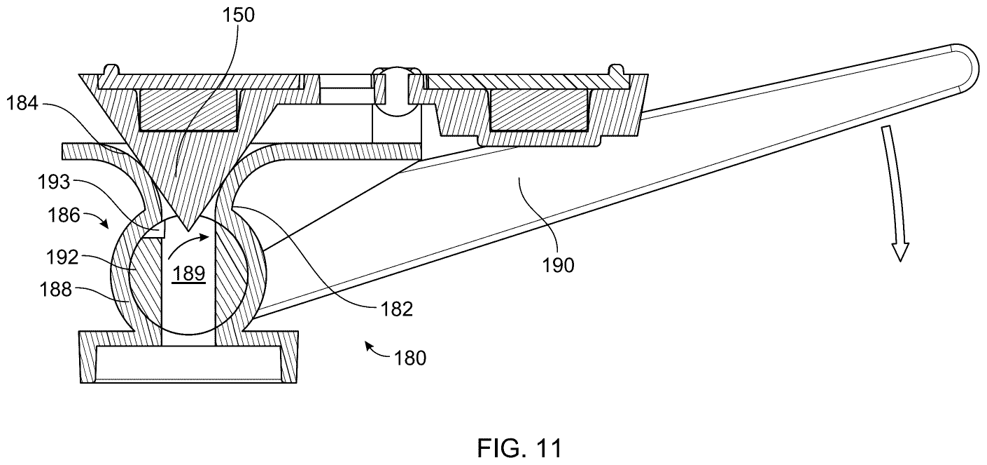

Referring to FIG. 11, a first cross-sectional view of the air manifold and oscillating mechanism of the positive expiratory pressure device with flow and oscillation control is shown.

The flow control mechanism 180 includes an air manifold 182 with conical exhalation port 184. Actuation of the flow control lever 190 causes rotation of the valve 186, specifically rotation of the valve ball 192 within the valve body 188.

A clearance slot 193 allows the rotation of the valve ball 192 to avoid contact with the conical plug 150.

The valve 186 is shown in its fully open position, allowing maximum air flow through the passageway 189 and thus through the air manifold 182.

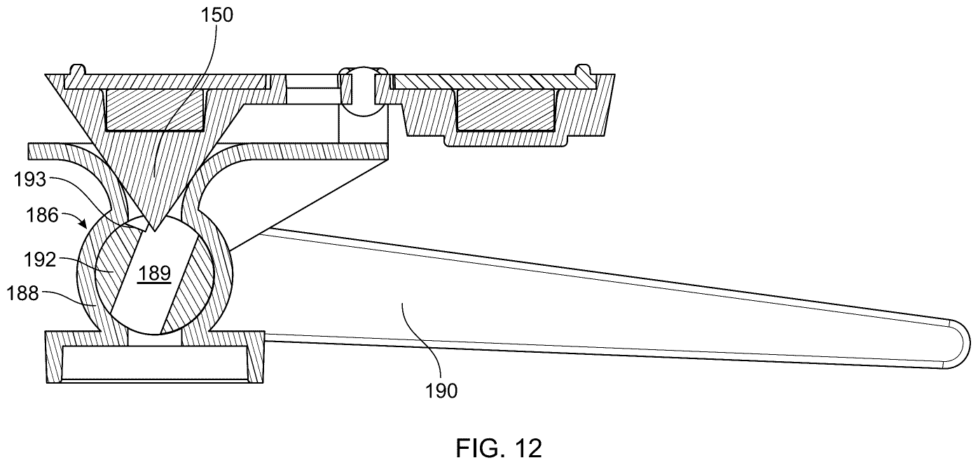

Referring to FIG. 12, a second cross-sectional view of the air manifold and oscillating mechanism of the positive expiratory pressure device with flow and oscillation control is shown.

In this figure the flow control lever 190 has caused rotation of the valve ball 192 within the valve body 188, restricting the cross-section of the passageway 189 within the valve ball 192 that is available for flow.

The tip of the conical plug 150 is within the clearance slot 193.

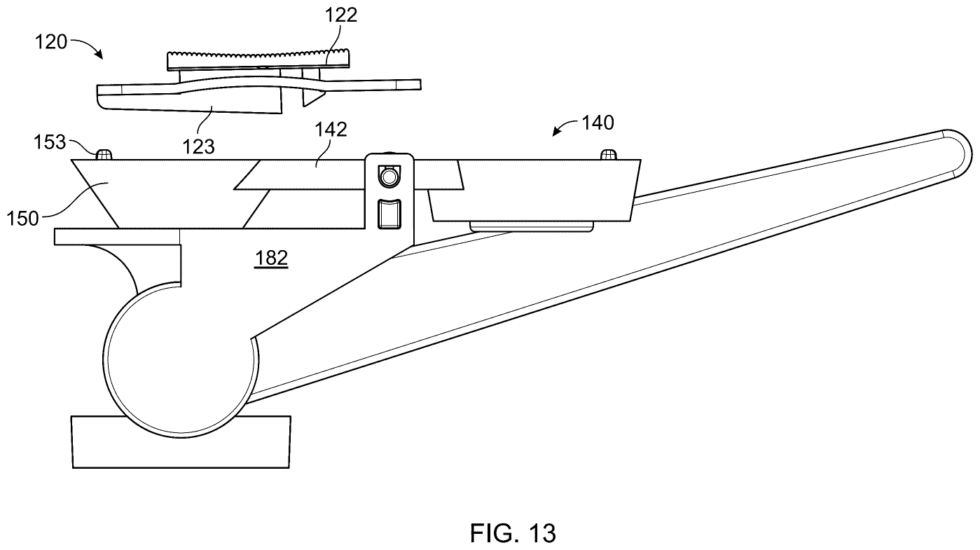

Referring to FIG. 13, a first view of the oscillating mechanism and frequency adjustment slider of the positive expiratory pressure device with flow and oscillation control is shown.

The oscillation frequency adjustment mechanism 120 allows for control and adjustment of the amplitude of motion of the oscillation mechanism 140. This is accomplished by movement of the slider 122 with respect to the oscillation mechanism 140, changing the position at which the first rocker arm cap protrusion 153 of the rocker arm 142 contacts with, and interacts with, the slider ramp 123, or angled ramp. As shown in FIG. 13, in its lowermost position, the conical plug 150 is fully seated within the air manifold 182.

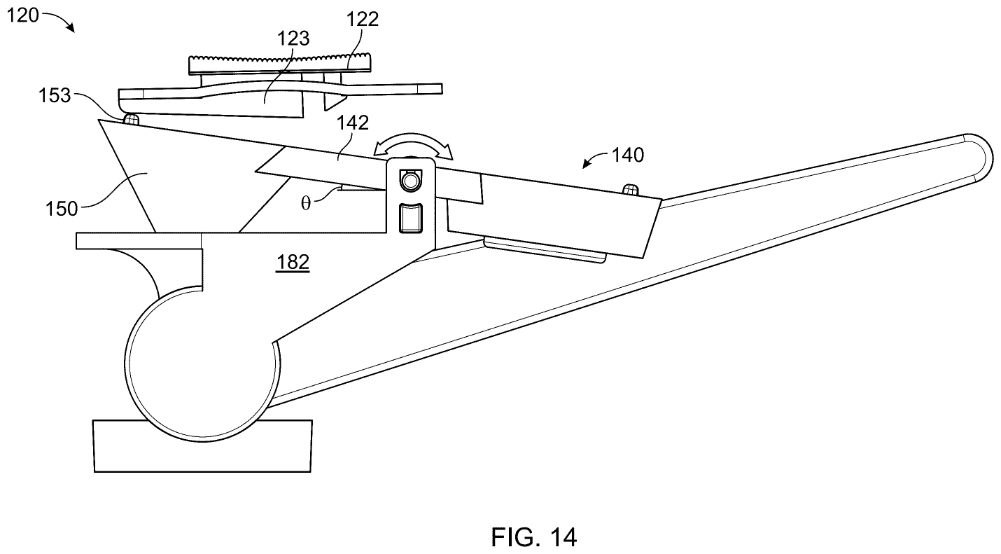

Referring to FIG. 14, a second view of the oscillating mechanism and frequency adjustment slider of the positive expiratory pressure device with flow and oscillation control is shown.

The oscillation frequency adjustment mechanism 120 is again shown with slider 122. Also shown is oscillation mechanism 140 and air manifold 182.

The rocker arm 142 is rotated, bringing the conical plug 150 upward and causing contact between the first rocker arm cap protrusion 153 and the slider ramp 123. At this contact point, the rocker arm is an angle Θ with respect to the original, seated position of the conical plug 150.

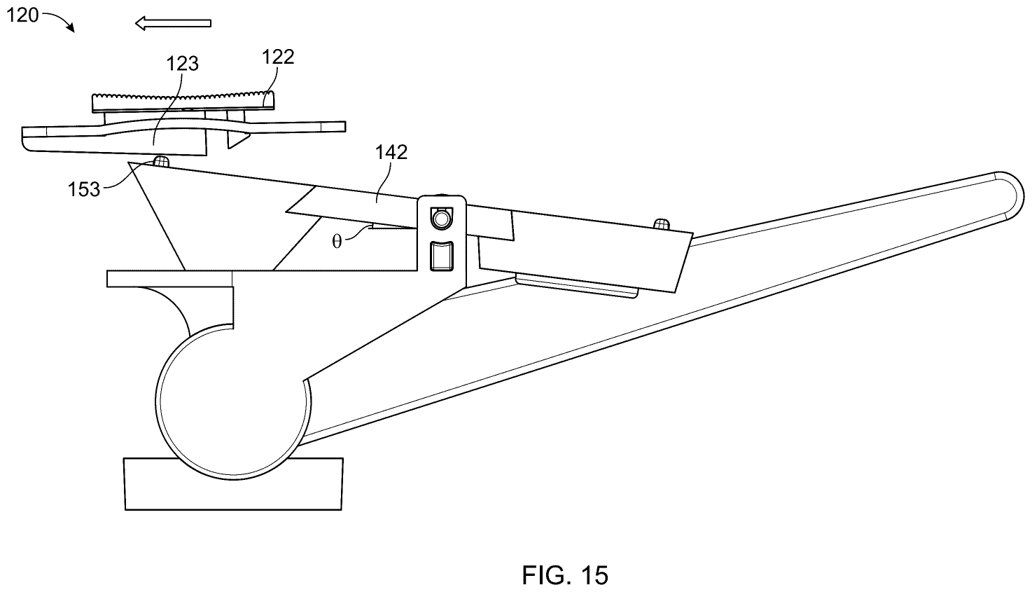

Referring to FIG. 15, a third view of the oscillating mechanism and frequency adjustment slider of the positive expiratory pressure device with flow and oscillation control is shown.

By moving the slider 122 of the oscillation frequency adjustment mechanism 120 to a different position, the angle Θ is reduced because the first rocker arm cap protrusion 153 contacts the slider ramp 123 at a lower angle Θ. By lowering the position of contact, the rocker arm 142 is permitted a lesser amount of rotation, or a decrease in amplitude. The result is an increased oscillation frequency.

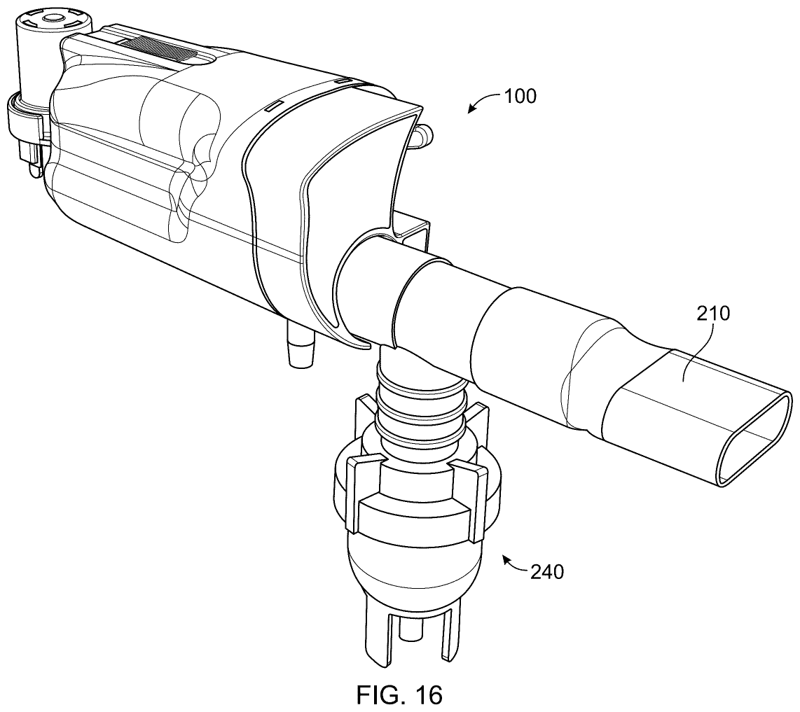

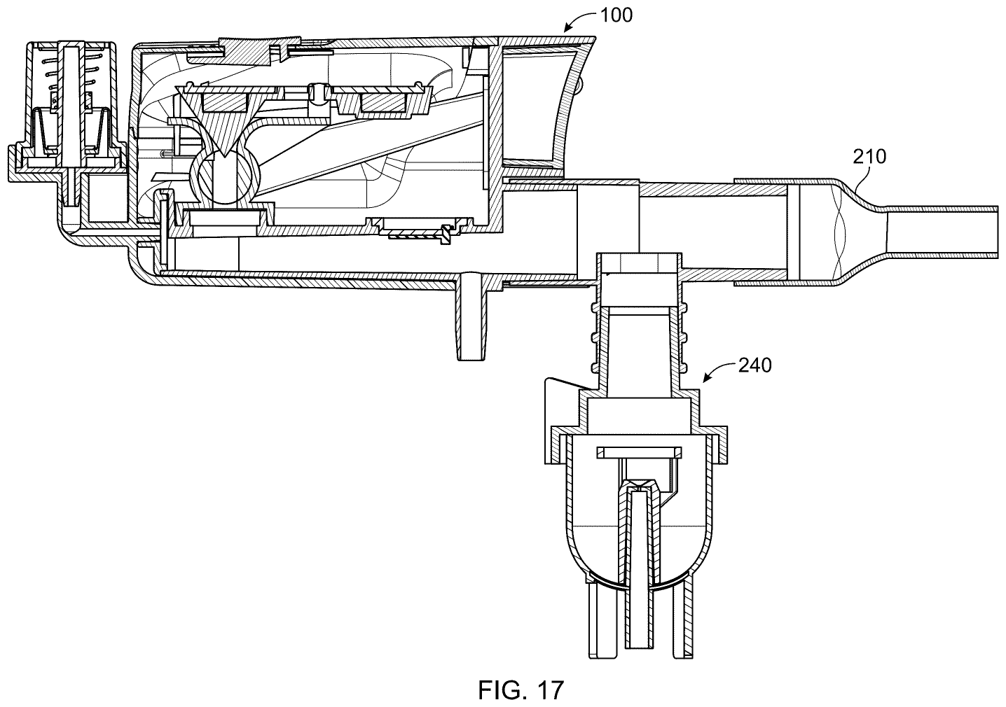

Referring to FIGS. 16 and 17, a second isometric view and a fourth cross-sectional view showing the optional nebulizer of the positive expiratory pressure device with flow and oscillation control are shown.

The positive expiratory pressure device with flow and oscillation control 100 is shown with optional nebulizer 240. Also shown is the mouth piece 210.

Equivalent elements can be substituted for the ones set forth above such that they perform in substantially the same manner in substantially the same way for achieving substantially the same result.

What is claimed is:

1. A positive expiratory pressure (PEP) device adaptable for a user with different breathing issues, comprising:
a housing defining an exhalation flow path;
an adjustable flow valve positioned along the exhalation flow path, the adjustable flow valve configured to control air flow resistance;
an oscillation mechanism positioned to interact with exhaled air in the exhalation flow path;
the oscillation mechanism repeatedly restricting and permitting air to flow; and
a slider control operatively connected to the oscillation mechanism;
a position of the slider control affecting a range of motion of the oscillation mechanism, thereby controlling oscillation frequency.

2. The positive expiratory pressure (PEP) device adaptable for a user with different breathing issues of claim 1, further comprising:
a mouthpiece;
a manometer;
the manometer located at a distal end of the housing with respect to the mouthpiece;
whereby placing the manometer at the distal end makes the manometer readily visible to a clinician.

3. The positive expiratory pressure (PEP) device adaptable for a user with different breathing issues of claim 1, further comprising:
an oxygen port placed along an inhalation flow path;
wherein a source of oxygen is optionally connected to the oxygen port, creating a device that provides oxygen to the user during inhalation and oscillating pressure changes during exhalation.

4. The positive expiratory pressure (PEP) device adaptable for a user with different breathing issues of claim 1, wherein the oscillation mechanism further comprises:
a rocker arm with a plugging end and a counterweight end;
the rocker arm pivoting across a fulcrum;
the plugging end moving toward and away from a conical exhalation port;
whereby as the plugging end moves in and out of the conical exhalation port, exhalation flow through the exhalation flow path is repeatedly stopped.

5. The positive expiratory pressure (PEP) device adaptable for a user with different breathing issues of claim 4, further comprising:
an oscillation frequency adjustment mechanism that controls an amplitude of motion of the rocker arm through interaction between an angled ramp and the rocker arm;
whereby contact between the rocker arm and different locations along the angled ramp changes the amplitude of motion, and therefore changing the oscillation frequency of the rocker arm.

6. The positive expiratory pressure (PEP) device adaptable for a user with different breathing issues of claim 4, wherein:
the adjustable flow valve is operable via a flow control lever, the flow control lever accessible outside of the housing of the device.

7. An oscillating positive expiratory pressure device for use with a wide range of users, comprising:
an exhalation pathway through a housing;
a flow control mechanism configured to adjust air flow resistance in the exhalation pathway;
an oscillation generating mechanism positioned in the exhalation pathway; and
an oscillation control mechanism operatively connected to the oscillation generating mechanism;
the oscillation control mechanism acting to adjust an oscillation frequency of the oscillation generating mechanism;
wherein the flow control mechanism and the oscillation control mechanism are independently adjustable to accommodate varying medical needs of different users.

8. The oscillating positive expiratory pressure device for use with a wide range of users of claim 7, whereby:
the oscillation control mechanism that controls an amplitude of motion of a rocker arm of the oscillation generating mechanism through interaction between an angled ramp and the rocker arm;
whereby contact between the rocker arm and different locations along the angled ramp changes the amplitude of motion, and therefore changing the oscillation frequency of the rocker arm.

9. The oscillating positive expiratory pressure device for use with a wide range of users of claim 7, further comprising:
a mouthpiece;
a manometer;
the manometer located at a distal end of the housing with respect to the mouthpiece;
whereby placing the manometer at the distal end makes the manometer readily visible to a clinician.

10. The oscillating positive expiratory pressure device for use with a wide range of users of claim 7, further comprising:
an oxygen port placed along an inhalation flow path;
wherein a source of oxygen is optionally connected to the oxygen port, creating a device that provides oxygen to a user during inhalation and oscillating pressure changes during exhalation.

11. The oscillating positive expiratory pressure device for use with a wide range of users of claim 7, wherein the oscillation generating mechanism further comprises:
a rocker arm with a plugging end and a counterweight end;
the rocker arm pivoting across a fulcrum;
the plugging end moving toward and away from a conical exhalation port;
whereby as the plugging end moves in and out of the conical exhalation port, exhalation flow through the exhalation pathway is repeatedly stopped.

12. The oscillating positive expiratory pressure device for use with a wide range of users of claim 7, wherein:
the flow control mechanism is operable via a flow control lever, the flow control lever accessible outside of the housing of the device.

13. The oscillating positive expiratory pressure device for use with a wide range of users of claim 11, further comprising:
an oscillation frequency adjustment mechanism that controls an amplitude of motion of the rocker arm through interaction of an angled ramp and the rocker arm;
whereby contact between the rocker arm and different locations along the angled ramp changes the amplitude of motion, and therefore changing the oscillation frequency of the rocker arm.

14. The oscillating positive expiratory pressure device for use with a wide range of users of claim 11, wherein:
the flow control mechanism is operable via a flow control lever, the flow control lever accessible outside of the housing of the device.

15. A positive expiratory pressure device for treating a variety of patient needs comprising:
a housing defining an exhalation flow path;
an exhalation flow control valve positioned in the exhalation flow path;
an oscillation mechanism configured to create pressure oscillations in exhaled air;
an oscillation frequency adjustment mechanism operatively connected to the oscillation mechanism;
wherein the exhalation flow control valve and the oscillation frequency adjustment mechanism are configured to cooperate, allowing for both flow and frequency adjustment during a user's exhalation, thereby accommodating a wide variety of patient needs.

16. The positive expiratory pressure device for treating a variety of patient needs of claim 15, further comprising:
a mouthpiece;
a manometer;
the manometer located at a distal end of the housing with respect to the mouthpiece;
whereby placing the manometer at the distal end makes the manometer readily visible to a clinician.

17. The positive expiratory pressure device for treating a variety of patient needs of claim 15, wherein:

18. The positive expiratory pressure device for treating a variety of patient needs of claim 15, further comprising:
an oxygen port placed along an inhalation flow path;
wherein a source of oxygen is optionally connected to the oxygen port, creating a device that provides oxygen to a user during inhalation and oscillating pressure changes during exhalation.

19. The positive expiratory pressure device for treating a variety of patient needs of claim 15, wherein the oscillation mechanism further comprises:
a rocker arm with a plugging end and a counterweight end;
the rocker arm pivoting across a fulcrum;
the plugging end moving toward and away from a conical exhalation port;
whereby as the plugging end moves in and out of the conical exhalation port, exhalation flow through the exhalation flow path is repeatedly stopped.

20. The positive expiratory pressure device for treating a variety of patient needs of claim 15, wherein:
the exhalation flow control valve is operable via a flow control lever, the flow control lever accessible outside of the housing of the device.
